(19) United States
(12) Patent Application Publication
(10) Pub. No.: US 2014/0223812 A1
(43) Pub. Date: Aug. 14, 2014

(54) Apparatus and Method for Dispensing Grains of Pollen

(71) Applicants: Jason Michael Cope, Ankeny, IA (US); Bradley Thomas McAvoy, West Des Moines, IA (US)

(72) Inventors: Jason Michael Cope, Ankeny, IA (US); Bradley Thomas McAvoy, West Des Moines, IA (US)

(73) Assignee: Pioneer Hi Bred International, Inc., Johnston, IA (US)

(21) Appl. No.: 14/006,715

(22) PCT Filed: Mar. 20, 2012
(86) PCT No.: PCT/US12/29743

§ 371 Date: Jan. 2, 2014

Related U.S. Application Data
(60) Provisional application No. 61/466,807, filed on Mar. 23, 2011.

(51) Int. Cl. A01H 1/02

(52) U.S. Cl. CPC A01H 1/025; A01H 1/02
USPC 47/1.41

(57) ABSTRACT

An apparatus configured for dispensing grains of pollen may include a pollen storage container, a flow source configured to dispense the grains of pollen from the pollen storage container, and a control mechanism configured to selectively activate the flow source. The apparatus may or may not apply an electrical charge to the grains of pollen. Related methods may include steps of collecting pollen, inserting the pollen into a pollen storage container, connecting the pollen storage container to a dispensing device such as the above-described apparatus, and dispensing the pollen from the pollen storage container with the dispensing device. Known grains of pollen may be dispensed to create predetermined gamete crosses with known plants. Further, an outlet of a fan which dispenses the pollen from the pollen storage container or other parts of the dispensing device may be cleaned when swapping the pollen storage container to prevent cross-contamination.

### FIELD OF THE INVENTION

Various embodiments of the present invention relate generally to methods and apparatuses for dispensing pollen. More specifically, embodiments of the present invention provide a method and apparatus configured to dispense pollen by dispensing pollen from a pollen storage container with a dispensing device.

### BACKGROUND OF THE INVENTION

For a variety of reasons, plant species may be intentionally bred. For example, in some applications plant species are intentionally bred to form hybrid plant species. In some applications, hybrid plants are bred to exhibit various desirable traits. Such traits may include, for example, resistance to heat and drought, resistance to disease and insect damage, improved yield characteristics, and improved agronomic quality. In general, plants may be capable of self-pollination, cross-pollination, or both. Self-pollination describes pollination using pollen from one flower that is transferred to the same or another flower of the same plant. Cross-pollination describes pollination using pollen delivered from a flower of a different plant from a different family or line.

Plants that have been self-pollinated and selected for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of heterogeneous plants that differ genetically and will not be uniform.

Maize (Zea mays L.), often referred to as corn in the United States, can be bred by both self-pollination and cross-pollination techniques. Maize has separate male and female flowers on the same plant. The male flowers are located on the tassel and the female flowers are located on the ear. Natural pollination occurs in maize when wind blows grains of pollen from the tassels to the silks that protrude from the tops of the ears.

The development of a hybrid maize variety in a maize seed production program may involve three steps: (1) the selection of plants from various germplasm pools for initial breeding crosses; (2) self-pollination of the selected plants from the breeding crosses for several generations to produce a series of inbred lines, which, individually breed true and are highly uniform; and (3) crossing a selected inbred line with an unrelated inbred line to produce the hybrid progeny. After a sufficient amount of inbreeding successive filial generations will merely serve to increase seed of the developed inbred. Preferably, an inbred line should comprise homozygous alleles at about 95% or more of its loci.

During the maize inbreeding process, vigor of the line may decrease. Vigor may be restored when two different inbred lines are crossed to produce the hybrid progeny. An important consequence of the homozygosity and homogeneity of the inbred lines is that the hybrid between a defined pair of inbreds may be reproduced indefinitely as long as the homogeneity of the inbred parents is maintained. Once the inbreds that create a superior hybrid have been identified, a continual supply of the hybrid seed can be produced using these inbred parents and the hybrid corn plants can then be generated from this hybrid seed supply.

Accordingly, development and production of maize seed may require controlled pollination at one or more steps, as described above.

### BRIEF SUMMARY

In one embodiment a method for dispensing grains of pollen is provided. The method may include obtaining a pollen storage container containing a plurality of grains of pollen to a dispensing device. Further, the method may include dispensing at least a portion of the plurality of grains of pollen from the pollen storage container with the dispensing device to pollinate a plant. At least one of the plant and the grains of pollen may be known.

In another embodiment a method for dispensing grains of pollen may include collecting a plurality of grains of pollen, inserting the plurality of grains of pollen into a pollen storage container, and connecting the pollen storage container containing the plurality of grains of pollen to a dispensing device. Further, the method may include dispensing at least a portion of the plurality of grains of pollen from the pollen storage container with the dispensing device to pollinate a plant. At least one of the plant and the grains of pollen may be known.

Further, the step of dispensing at least a portion of the plurality of grains of pollen may comprise dispensing at least a portion of the plurality of grains of pollen without applying an electrical charge to the portion of the plurality of grains of pollen. In another embodiment the method may include applying an electrical charge to the portion of the plurality of grains of pollen. The method may also include providing for grounding a user of the dispensing device.

Additionally, the method may comprise controlling dispensing of the portion of the plurality of grains of pollen by selectively varying a flow rate at which the dispensing device dispenses the plurality of grains of pollen. Also, the method may include varying the flow rate by adjusting a fan speed of the dispensing device. The method may further comprise powering the dispensing device with a battery.

Also, the method may include mixing the grains of pollen with an additive prior to dispensing the grains of pollen. The additive may be configured to enhance visibility of the plurality of grains of pollen dispensed from the dispensing device. The additive may additionally or alternatively be configured to control a moisture level of the plurality of grains of pollen. Further, the step of pollinating the plant with the portion of the plurality of known grains of pollen may comprise pollinating a corn plant. The method may further include preventing pollination by an alternate pollen source by covering a plurality of silks of the corn plant prior to pollinating the corn plant with the dispensing device.

The method may additionally comprise controlling an environmental condition within the pollen storage container. The environmental condition may comprise temperature in some embodiments. In a further embodiment the environment condition may comprise humidity. Also, the method may include dispensing at least a portion of a second plurality of grains of pollen from a second pollen storage container with the dispensing device. Further, the method may comprise cleaning the dispensing device prior to dispensing the portion of the second plurality of grains of pollen. Cleaning the dispensing device may comprise cleaning an outlet of a fan of the dispensing device.

In an additional embodiment a method for dispensing grains of pollen is provided. The method may comprise collecting a plurality of grains of pollen, inserting the plurality of grains of pollen into a pollen storage container, connecting the pollen storage container containing the plurality of grains of pollen to a dispensing device, and dispensing at least a portion of the plurality of grains of pollen from the pollen storage container with the dispensing device without applying an electrical charge to the portion of the plurality of grains of pollen.

In another embodiment an apparatus configured for dispensing grains of pollen is provided. The apparatus may comprise a pollen storage container configured to receive a plurality of grains of pollen, a flow source configured to dispense at least a portion of the plurality of know grains of pollen from the pollen storage container, and a control mechanism configured to selectively activate the flow source. The apparatus may not be configured to apply an electrical charge to the portion of the plurality of grains of pollen in some embodiments. Further, the apparatus may include an environmental control mechanism configured to control an environmental condition within the pollen storage container.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates an embodiment of an apparatus configured for dispensing grains of pollen in accordance with an example embodiment of the present invention;

FIG. 2 illustrates a pollen storage container of the apparatus of FIG. 1 in accordance with an example embodiment of the present invention;

FIG. 3 illustrates an embodiment of a method for dispensing grains of pollen without applying an electrical charge to the grains of pollen in accordance with an example embodiment of the present invention; and

FIG. 4 illustrates a method for dispensing grains of pollen in accordance with an example embodiment of the present invention.

### DETAILED DESCRIPTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Controlled pollination of plants, for example during cross-pollination, may involve manually collecting and dispensing pollen. Manually collecting and dispensing pollen may be very labor intensive. For example, a worker may manually collect pollen by knocking it off of the tassels of the ear corn into a tassel bag. Further, the worker may then manually sprinkle the pollen onto the anthers of female corn which have been covered with a shoot bag so as to prevent pollination with pollen other than the collected grains of pollen. Thus, collection of pollen and controlled pollination using the collected pollen may involve many manual steps that may be conducted over the course of one or more days. Further, application of pollen using tassel bags may not efficiently make use of the collected pollen since the tassel bag may not allow for targeted application of the pollen in an easily controlled manner. Thus, workers may tend to apply more of the pollen than would otherwise be necessary. Accordingly, embodiments of the invention provide improved apparatuses and methods for dispensing pollen.

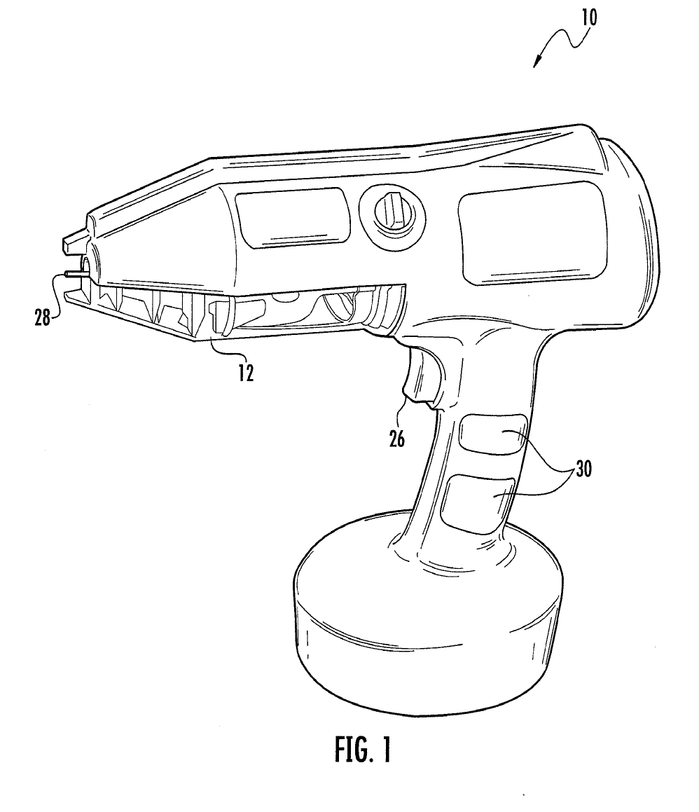

In this regard, FIG. 1 illustrates an embodiment of an apparatus 10 configured for dispensing grains of pollen. The apparatus 10 may comprise a pollen storage container 12 configured to receive a plurality of grains of pollen. As will be described below, in some embodiments the grains of pollen may be known. Further, the apparatus 10 may include a flow source such as a fan or compressed air source that is configured to dispense at least a portion of the plurality of grains of pollen from the pollen storage container 12. However, dispensing the grains of pollen may employ various other embodiments of mechanisms and methods for imparting motion to the grains of pollen.

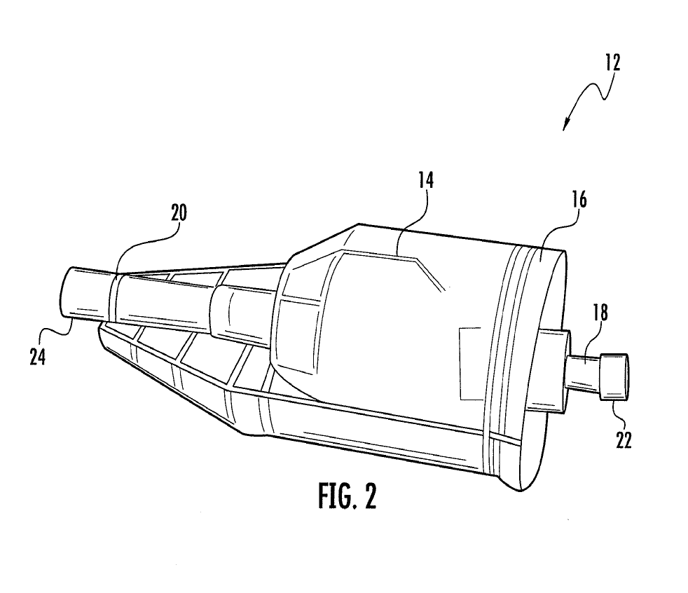

FIG. 2 illustrates an enlarged view of the pollen storage container 12. In this regard, the pollen storage container 12 may be removable from the apparatus 10. As illustrated, the pollen storage container 12 may comprise a compartment 14 and a lid 16. The lid 16 may be removable so that the compartment 14 may receive the grains of pollen. Further, removal of the lid 16 may facilitate cleaning of the compartment 14 in embodiments in which the pollen storage container 12 is reusable.

The pollen storage container 12 may further comprise an inlet 18 and an outlet 20. The inlet 20 of the pollen storage container 12 may be provided in the lid 16 in some embodiments. Further, the inlet 18 may be configured to receive an air flow (or other fluid flow) from a flow source. As described above, the apparatus 10 may comprise a fan which blows air. Thus, an outlet of the fan may be in fluid communication with the inlet 18 of the pollen storage container 12. Thereby, the flow of air may agitate or otherwise impart motion to the grains of pollen in the compartment 14 so as to dispense at least a portion of the grains of pollen through the outlet 20 of the pollen storage container 12.

The grains of pollen may be dispensed from the apparatus 10 directly from the outlet 20 of the pollen storage container 12 in some embodiments. Thus, the potential for cross-contamination within the apparatus 10 may be reduced. For example, the apparatus 10 may successively or simultaneously connect to multiple pollen storage containers 12 to dispense different types of pollen, or a single pollen storage container may be cleaned and reused with multiple types of pollen. By storing and dispensing the grains of pollen with the pollen storage container 12, the grains of pollen may substantially avoid contact with the remainder of the apparatus 10. The outlet of the fan or other flow source which is in fluid communication with the inlet 18 of the pollen storage container 12 may present one of the few potential areas where cross-contamination may occur. Thus, the outlet of the fan or other flow source may be cleaned when the pollen storage container 12 is removed between successive uses of different types of pollen.

The pollen storage containers 12 may be employed to store the grains of pollen prior to dispensing the grains of pollen using the apparatus 10. In this regard, inlet 22 and outlet 24 plugs may be employed to seal the inlet 18 and the outlet 20 such that the grains of pollen are sealed within the compartment 14. In some embodiments the inlet plug 22 and the outlet 24 plug may comprise silicone, although other materials may be employed in other embodiments as may be understood by one having skill in the art.

As illustrated in FIG. 1, the apparatus 10 may further comprise a control mechanism such as a trigger 26. The trigger 26 (or other control mechanism) may be configured to selectively activate the flow source. In some embodiments the trigger 26 (or other control mechanism) may be configured to selectively vary a flow rate at which the apparatus 10 dispenses the grains of pollen. For example, the flow rate may increase with increasing displacement of the trigger 26 as the trigger is depressed.

In some embodiments, the apparatus 10 may be configured to apply an electrical charge to the portion of the plurality of grains of pollen that are dispensed. In the depicted embodiment, an electrode 28 may be positioned near the outlet 20 of the pollen storage container 12. The electrode 28 may be configured to impart a charge on the grains of pollen as they are dispensed through the outlet 20 of the pollen storage container 12. The electrical charge imparted by the electrode 28 may be, for example, configured to attract the grains of pollen to silks of the corn to which the grains of pollen are applied. Accordingly, applying an electrical charge to the pollen may facilitate pollination. However, not all embodiments of the apparatus 10 may apply an electrical charge to the grains of pollen, and pollination may occur without application of an electrical charge.

In embodiments of the apparatus 10 employing the electrode 28 to apply an electrical charge to the grains of pollen, the apparatus may further comprise contacts 30 (or other device) configured to ground a user of the apparatus. For example, the contacts 30 may be configured to ground the user and/or the apparatus 10 through a ground line coupled to a ground prong of an electrical outlet in embodiments employing an electrical cord. Thereby, the ground may prevent a charge from building on the user or the apparatus 10 as the electrode 28 imparts a charge to the grains of pollen.

The above-described apparatus 10 may comprise a powder coating gun sold as Model No. 01-06100 by WAI Powder Coating System, of Royersford, Pa., or an adaptation thereof. For example, the electrode 28 may be removed in embodiments of the apparatus 10 configured to dispense the grains of pollen without applying an electrical charge to the grains of pollen. Further, the apparatus 10 may be adapted to be powered by a battery as opposed to powered by an electrical cord.

Additionally, the apparatus 10 may further comprise an environmental control mechanism configured to control an environmental condition within the pollen storage container 12. For example, the environmental condition may comprise temperature, humidity, light exposure, and/or other environmental conditions. Thereby, the apparatus 10 may comprise a heating element, condenser, or other environmental control mechanism configured to heat or cool the grains of pollen in the pollen storage container 12. In one embodiment the environmental control mechanism may comprise a jacket configured to partially or fully surround the pollen storage container 12. The jacket may be heated or cooled so as to control the temperature within the pollen storage container 12 to achieve a desired temperature of the grains of pollen therein and thereby maintain viability of the grains of pollen. In one embodiment the jacket may comprise a gel or other substance of relatively high thermal capacity. Thereby, for example, the jacket may be heated or cooled, and then attached to the pollen storage container 12 to heat or cool the grains of pollen therein without necessarily requiring a power source to control the temperature of the grains of pollen during the usage of the apparatus 10. Further a fan, the condenser, or a separate dehumidifier may be employed to regulate humidity within the compartment 14 of the pollen storage container.

With regard to light exposure, a coating may be applied to the pollen storage container 12 to reduce visible, ultraviolet, and/or infrared light transmission through the pollen storage container to the grains of pollen. Alternatively, the material from which the pollen storage container is formed may be selected to limit light transmission. Accordingly, one or more conditions within the compartment 14 of the pollen storage container 12 may be regulated. Thereby, for example, the viability of the grains of pollen may be maintained for an extended period of time. Alternatively or additionally, the environmental conditions may be controlled in a manner that facilitates dispensing of the grains of pollen and/or improves adhesion of the grains of pollen to the plant to which the grains of pollen are applied.

However, various other embodiments of apparatuses may be employed to dispense pollen by dispensing grains of pollen from a pollen storage container beyond the embodiment illustrated in FIGS. 1 and 2 and described above. For example, alternate embodiments of apparatuses configured for dispensing pollen may include powder coating apparatuses compressed air apparatuses, and various other devices configured or adapted to dispense grains of pollen. Thus, dispensing grains of pollen may comprise dispensing the grains of pollen by dispersing, atomizing, blowing, ionizing, or any other mechanism or method for imparting motion to the grains of pollen beyond that caused exclusively by manual displacement and/or gravitational force. Depending on the particular embodiment of the apparatus employed, the pollen storage container may comprise a vial, vessel, compartment, or various other structures configured to hold the grains of pollen.

Thus, various embodiments of apparatuses may be employed to dispense the grains of pollen. Methods for dispensing grains of pollen will now be described. While the description may generally describe operations conducted by embodiments of the apparatus 10 illustrated in FIGS. 1 and 2, it should be understood that this is for purposes of brevity only. In this regard, various other ones of the above-described apparatuses may be employed to conduct the methods that will be described below.

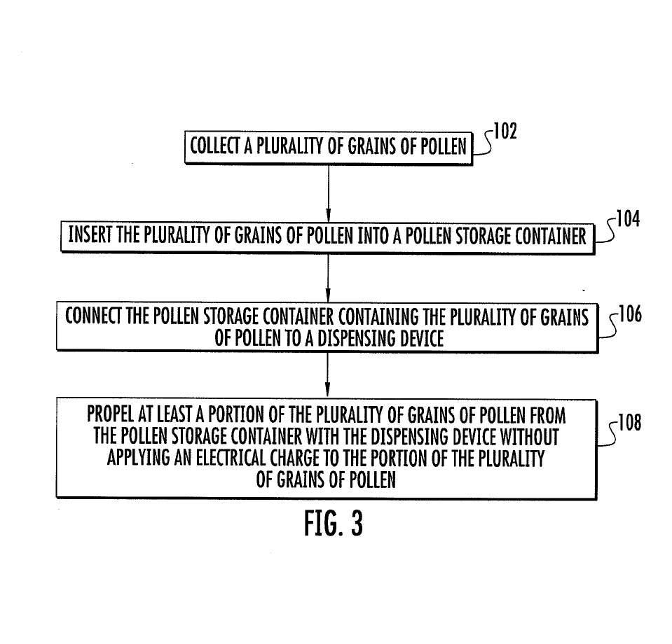

FIG. 3 illustrates an embodiment of a method for dispensing grains of pollen. As illustrated, the method may comprise collecting a plurality of grains of pollen at operation 102. Collecting pollen at operation 102 may comprise knocking pollen off of tassels of ear corn into a tassel bag, vacuuming the pollen off of the tassels, or any other method of collecting pollen as may be understood by one having skill in the art. The method may further comprise inserting the plurality of grains of pollen into a pollen storage container at operation 104. For example, the method may employ the pollen storage container 12 to hold the pollen, and the lid 16 of the pollen storage container may be removed to facilitate insertion of the grains of pollen as described above. However, various other pollen storage containers may be employed in other embodiments of the method.

The method may also comprise connecting the pollen storage container containing the plurality of grains of pollen to a dispensing device at operation 106. For example, the above-described pollen storage container 12 of FIG. 2 may be connected to the apparatus 10, as illustrated in FIG. 1. However, as noted above, various other embodiments of dispensing devices may be employed. In some embodiments the pollen may be stored prior to connecting the pollen storage container containing the grains of pollen to the dispensing device at operation 106. In some embodiments the pollen may be stored in the pollen storage container 12, as described above. In other embodiments the pollen may additionally or alternatively be stored prior to inserting the pollen into the pollen storage container at operation 104.

The method may also comprise dispensing at least a portion of the plurality of grains of pollen from the pollen storage container with the dispensing device without applying an electrical charge to the portion of the plurality of grains of pollen at operation 108. In this regard, in embodiments employing the apparatus 10 of FIG. 1, the electrode 28 may be removed or disabled so as to not apply an electrical charge to the grains of pollen. By dispensing the grains of pollen in a targeted and controlled manner, use of electrostatic attraction caused by applying an electrical charge to the grains of pollen may not be needed. Thus, use of the illustrated contacts 30 may not be necessary, because there may be no need to ground the user of the apparatus 10 or the apparatus itself. Accordingly, a battery may be used to power the apparatus 10, because a ground wire may not be necessary and hence the apparatus may become more portable without the use of power and ground cords.

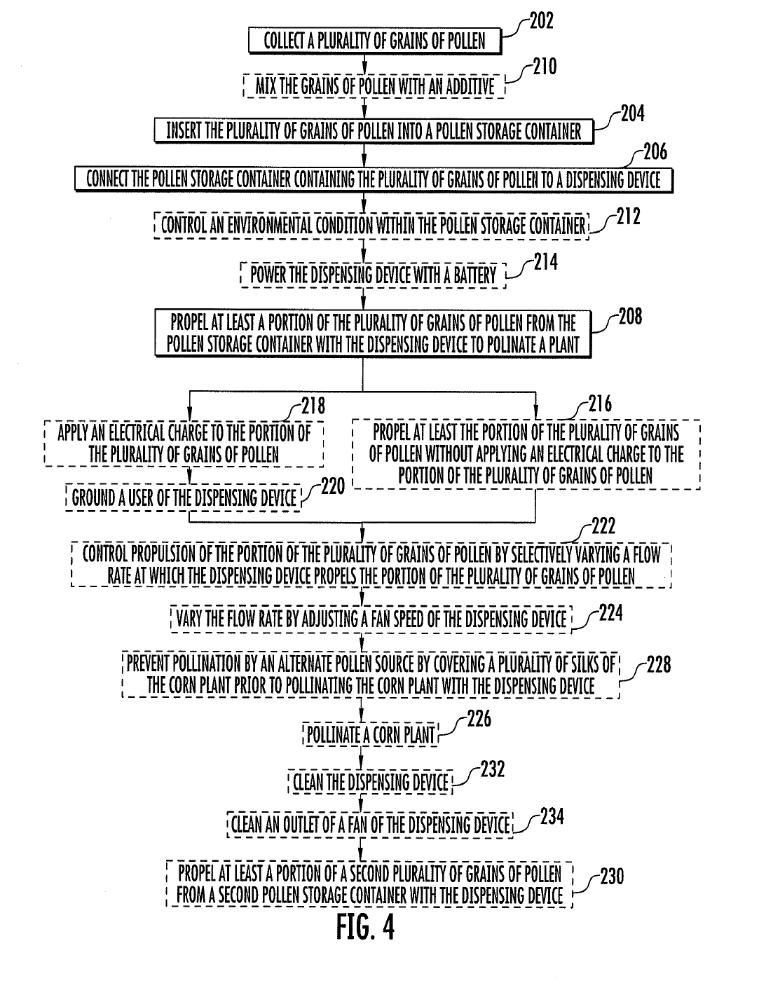

FIG. 4 illustrates an additional embodiment of a method for dispensing grains of pollen. Various ones of the operations depicted in the method of FIG. 3 and the method of FIG. 4 may be interchangeable between the two methods. In particular, FIG. 4 depicts a method for dispensing grains of pollen to pollinate a plant. The plant and/or the grains of pollen may be known. Known grains of pollen may comprise grains of pollen for which characteristics of the grains of pollen are known. For example, the specific plant from which the grains of pollen are collected may be known, the genetic make-up of the grains of pollen may be known, and/or the plot from which the grains of pollen are collected may be known. Similarly, a known plant may refer to a plant for which the genetic make-up or other characteristics of the plant are known

As illustrated, the method may comprise collecting a plurality of grains of pollen at operation 202. Further, the method may comprise inserting the plurality of grains of pollen into a pollen storage container at operation 204. Additionally, the method may include connecting the pollen storage container containing the plurality of grains of pollen to a dispensing device at operation 206. Also, the method may comprise dispensing at least a portion of the plurality of grains of pollen from the pollen storage container with the dispensing device to pollinate a plant at operation 208. As described above, at least one of the plant and the grains of pollen may be known in some embodiments.

In some embodiments the method may additionally or alternatively comprise other operations including those operations illustrated in dashed lines in FIG. 4. For example, the method may include mixing the grains of pollen with an additive at operation 210, which may occur prior to dispensing the grains of pollen at operation 208. The additive may be configured to enhance visibility of the plurality of grains of pollen dispensed from the dispensing device in some embodiments. For example, the additive may comprise a white talcum powder, or other substance that is easily visible. Thereby, the operator may be able to see the flow rate of the grains of pollen as they are dispensed from the pollen storage container and further the operator may see the amount of pollen that lands on the plant (as represented by the additive). For example, the grains of pollen and additive may be mixed in a one-to-one ratio (by mass or volume) in some embodiments. The additive may additionally or alternatively be configured to control a moisture level of the plurality of grains of pollen. For example, the additive may increase the duration of viability of the grains of pollen by providing moisture to the grains of pollen. In this regard, the additive may store and release moisture.

Additionally, the method may comprise controlling an environmental condition within the pollen storage container at operation 212. As described above, in some embodiments the environmental condition may comprise temperature, humidity, and/or light exposure in some embodiments, although various other environmental conditions may be controlled in other embodiments. The method may also include powering the dispensing device with a battery, as illustrated at operation 214. However, in other embodiments the dispensing device may be powered through use of a power cord, which may, for example, receive and convert alternating current to direct current.

Further, the step of dispensing at least a portion of the plurality of grains of pollen from the pollen storage container at operation 208 may comprise dispensing at least a portion of the plurality of grains of pollen without applying an electrical charge to the portion of the plurality of grains of pollen at operation 216. Although the method in FIG. 3 described above related exclusively to embodiments in which an electrical charge was not applied to the grains of pollen, this may not be the case for the method depicted in FIG. 4. In this regard, as illustrated at operation 218, the method may comprise applying an electrical charge to the portion of the plurality of grains of pollen. Thereby, the binding potential of the grains of pollen with the plant may be increased. The method may also comprise providing for grounding a user of the dispensing device at operation 220 so as to prevent the build-up of a charge on the user/and or dispensing device, as described above. For example, the user may ground through the contacts 30 on the apparatus 10.

The method may additionally comprise controlling dispensing of the portion of the plurality of grains of pollen by selectively varying a flow rate at which the dispensing device dispenses the plurality of grains of pollen at operation 222. In some embodiments varying the flow rate at operation 222 may comprise adjusting a fan speed of the dispensing device at operation 224. For example, in embodiments employing the apparatus 10, the trigger 26 may be configured to vary the fan speed depending on how far the trigger is depressed.

Additionally, pollinating the plant with the portion of the plurality of grains of pollen may comprise pollinating a corn plant at operation 226, as mentioned above in an example embodiment. However, as may be understood by one having skill in the art, the methods and apparatuses disclosed herein may be employed to pollinate various other varieties of plants. In embodiments of the method involving pollinating a corn plant at operation 226, the method may further comprise preventing pollination by an alternate pollen source by covering a plurality of silks of the corn plant prior to pollinating the corn plant with the dispensing device, as illustrated at operation 228. Thereby, it may be more likely that pollination occurs with the grains of pollen dispensed from the pollen storage container rather than with other grains of pollen from alternate pollen sources.

In some embodiments the method may further comprise dispensing at least a portion of a second plurality of grains of pollen from a second pollen storage container with the dispensing device at operation 230. The second pollen storage container may be a separate pollen storage container in some embodiments, whereas in other embodiments the second pollen storage container may refer to reuse of the first pollen storage container after the first plurality of grains of pollen have been removed and the second plurality of grains of pollen have been inserted. Thus, a single pollen storage container may be reused in some embodiments, whereas in other embodiments new pollen storage containers may be employed. The pollen storage container may be cleaned when reused to prevent cross-contamination in some embodiments.

The method may also comprise cleaning the dispensing device at operation 232 prior to dispensing the portion of the second plurality of grains of pollen at operation 230. Cleaning the dispensing device at operation 232 may involve cleaning an outlet of a fan of the dispensing device at operation 234. However, various other parts and components of the dispensing device may be cleaned in other embodiments. For example, in embodiments employing the apparatus 10 to apply an electrical charge to the grains of pollen, the electrode 28 may also be cleaned. In this regard, it is noted that embodiments of the apparatus 10 which do not employ the electrode 28 to apply an electric charge to the grains of pollen may present fewer potential possibilities for cross-contamination.

By employing the methods and/or the apparatus 10 disclosed herein, the grains of pollen may be applied in a relatively more efficient manner than may be accomplished by manually applying the pollen with a tassel bag. In particular, the apparatus 10 (or other dispensing device in accordance with the disclosure herein) may selectively dispense the pollen onto the plant in a controlled manner. Further, the pollen may be dispensed directly at the desired portion of the plant so as to more effectively use the grains of pollen, and potentially lead to greater pollination rates. By selectively directing pollen in this manner, less pollen may be required to pollinate the same number of plants as compared to application of the pollen using tassel bags. Additionally, selectively directing pollen in this manner may cause the pollen to pollinate only the target plant, since the direction in which the pollen are directed and the quantity thereof may be carefully controlled using a dispensing device such as the apparatus 10 from FIG. 1.

In some embodiments pollinating the plant with the portion of the plurality of grains of pollen may comprise pollinating a known plant with known grains of pollen to create a predetermined gamete cross. Thus, knowledge of the characteristics of the pollen and the plant to which the pollen is applied may be employed to create a known cross in some embodiments. However, in other embodiments pollinating the plant with the portion of the plurality of grains of pollen may comprise pollinating an unknown plant and/or pollinating a plant with plurality of unknown grains of pollen to create a gamete cross. In this regard, in some embodiments the characteristics of the cross may not be completely known at the time of pollination.

Many modifications and other embodiments of the invention set forth herein will come to mind to one skilled in the art to which these invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for dispensing grains of pollen, comprising:
obtaining a pollen storage container containing a plurality of grains of pollen to a dispensing device; and
dispensing at least a portion of the plurality of grains of pollen from the pollen storage container with the dispensing device to pollinate a plant,
wherein at least one of the plant and the grains of pollen are known.

2. The method of claim 1, wherein the step of dispensing at least a portion of the plurality of grains of pollen comprises dispensing at least a portion of the plurality of grains of pollen without applying an electrical charge to the portion of the plurality of grains of pollen.

3. The method of claim 1, further comprising applying an electrical charge to the portion of the plurality of grains of pollen.

4. The method of claim 3, further comprising providing for grounding a user of the dispensing device.

5. The method of claim 1, further comprising controlling dispensing of the portion of the plurality of grains of pollen by selectively varying a flow rate at which the dispensing device dispenses the plurality of grains of pollen.

6. The method of claim 5, further comprising varying the flow rate by adjusting a fan speed of the dispensing device.

7. The method of claim 1, further comprising powering the dispensing device with a battery.

8. The method of claim 1, further comprising mixing the grains of pollen with an additive prior to the step of dispensing the grains of pollen.

9. The method of claim 8, wherein the additive is configured to enhance visibility of the plurality of grains of pollen dispensed from the dispensing device.

10. The method of claim 8, wherein the additive is configured to control a moisture level of the plurality of grains of pollen.

11. The method of claim 1, wherein the step of pollinating the plant with the portion of the plurality of known grains of pollen comprises pollinating a corn plant.

12. The method of claim 11, further comprising preventing pollination by an alternate pollen source by covering a plurality of silks of the corn plant prior to pollinating the corn plant with the dispensing device.

13. The method of claim 1, further comprising controlling an environmental condition within the pollen storage container.

14. The method of claim 13, wherein the environmental condition comprises temperature.

15. The method of claim 13, wherein the environment condition comprises humidity.

16. The method of claim 1, further comprising dispensing at least a portion of a second plurality of grains of pollen from a second pollen storage container with the dispensing device.

17. The method of claim 16, further comprising cleaning the dispensing device prior to dispensing the portion of the second plurality of grains of pollen.

18. The method of claim 17, wherein cleaning the dispensing device comprises cleaning an outlet of a fan of the dispensing device.

19. A method for dispensing grains of pollen, comprising:
Obtaining a pollen storage container containing a plurality of grains of pollen to a dispensing device; and
dispensing at least a portion of the plurality of grains of pollen from the pollen storage container with the dispensing device without applying an electrical charge to the portion of the plurality of grains of pollen.

20. An apparatus configured for dispensing grains of pollen, comprising:
a pollen storage container configured to receive a plurality of grains of pollen;
a flow source configured to dispense at least a portion of the plurality of know grains of pollen from the pollen storage container; and
a control mechanism configured to selectively activate the flow source,
wherein the apparatus is not configured to apply an electrical charge to the portion of the plurality of grains of pollen.

21. The apparatus of claim 20, further comprising an environmental control mechanism configured to control an environmental condition within the pollen storage container.
